(19) United States
(12) Patent Application Publication
(10) Pub. No.: US 2008/0032926 A1
(43) Pub. Date: Feb. 7, 2008

(54) Knockout Non-Human Animal

(76) Inventors: Yoshikazu Naito, Toyonaka-shi (JP); Kenji Oeda, Kyoto (JP); Tokuo Sukata, Osaka-shi (JP); Yoshifumi Inoue, Suita-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/659,807

(22) PCT Filed: Aug. 9, 2005
(86) PCT No.: PCT/JP05/14881

§ 371 Date: Feb. 9, 2007

(30) Foreign Application Priority Data
Aug. 10, 2004 (JP) 2004-233089

(51) Int. Cl. A61K 38/00; A01K 67/00; A61K 48/00; C12N 15/00; C12Q 1/68; G01N 33/00; C12N 5/06; C07K 16/18; A01K 67/027

(52) U.S. Cl. 514/12; 435/320.1; 435/325; 435/6; 514/1; 514/44; 530/387.9; 800/14; 800/18; 800/3; 800/9

(57) ABSTRACT

The present invention relates to a knockout non-human animal whose genome comprises a disruption in at least one allele of the PHF3 gene, useful in developing drugs for diseases with the onset of atopic dermatitis or the like; a totipotent cell such as an ES cell which is essential to production of the above non-human animal; use thereof; and so on.

### TECHNICAL FIELD

The present invention relates to a knockout non-human animal in which the PHF3 gene is disrupted and the like.

### BACKGROUND ART

Allergic diseases including atopic dermatitis, asthma, allergic rhinitis, allergic conjunctivitis and the like cause hypersensitive responses to an environmental antigen and the like to which normal people do not respond, and lead to failure of each organ via an immunoglobulin E mediated inflammatory mechanism.

There are a variety of genome regions involved in the development of allergic diseases, and there are many disease-causative genes corresponding to the genome regions (Halapi et al., Curr Opin Pulm Med., 10:22-30, 2004; Lee et al., Nat Genet., 26:470-473, 2000; Cookson et al., Lancet, 1:1292-1295, 1989; Bradley et al., Hum. Mol. Genet., 11;1539-1548, 2002). In some of genome regions involved in allergic diseases, a disease-causative gene and its physiological mechanism have been known. For example, it has been known that the genome region 5q31 encodes the cytokine IL-4 gene and mutation of said gene is associated with severity of atopic dermatitis (Bradley et al., Hum. Mol. Genet., 11;1539-1548, 2002; Novak et al., J. Invest. Dermatol., 119:870-875, 2002). Secretion of so-called Th2 cytokines such as IL-4, 5, 13 and the like is promoted by activation of helper T2 cells. These cytokines stimulate B cells to induce an allergic reaction accompanied by an increased expression of immunoglobulin E.

Among model animals for atopic dermatitis, a NC/Nga mouse, a NOA mouse, a C57BL/Ka cpdm/cpdm mouse and a Ds-Nh mouse draw attention as spontaneous atopic dermatitis model animals (Atsuko Itakura et al., SAISHIN IGAKU, vol. 53: 2848-2854, 1998; Mouse Genome, 95:698-700, 1997; HogenEsch et al., Am J Pathol., 143:972-982, 1993; Hikita et al., J Dermatol Sci., 30:142-153, 2002). In these model animals, mutant genes have not been identified. In addition, a TNP-IgE transgenic mouse, a IL-18 transgenic mouse, a Caspase-1 transgenic mouse and a cathepsin E knockout mouse which are produced by gene alteration are also known as model animals for atopic dermatitis (Matsuoka et al., Int Immunol., 11:987-994, 1999; Yoshimoto et al., Nat Immunol., 1:132-137, 2000; Yamanaka et al., J Immunol., 165:997-1003, 2000; Tsukuba et al., J Biochem (Tokyo)., 134:893-902, 2003).

The gene of the PHF3 protein has been cloned as a gene whose expression is reduced in human gliomas (Fischer et al., Cytogenet Cell Genet., 94 :131-136, 2001). The protein is characterized by possession of a PHD domain (a type of zinc finger domain) and therefore has been called PHD finger domain 3.

### DISCLOSURE OF INVENTION

An object of the present invention is to provide a knockout non-human animal and the like in which a novel causative gene is disrupted, useful in developing remedies and the like for diseases with the development of atopic dermatitis.

The present inventors disrupted the PHF3 gene on a chromosome of a mouse ES cell, mixed the resulting ES cell with the blastocyst of a parent animal, allowed embryonic differentiation in the parent animal to continue until a baby was born, and thereby a chimeric primitive mouse was produced as an offspring. Then, the present inventors bred the chimeric primitive mouse to produce a progeny (hereinafter, the chimeric primitive mouse and a progeny thereof are referred to as the present knockout mouse in some cases). In the present knockout mouse, scab formation and cell infiltration into the skin dermis layer were high-frequently observed, and the total immunoglobulin E level in blood was extremely high. Based on these findings, the present inventors found that said animal can be a model mouse representing disease states similar to the development of atopic dermatitis, and as a result, completed the present invention.

That is, the present invention provides:

1. A knockout non-human animal whose genome comprises a disruption in at least one allele of the PHF3 gene (hereinafter, referred to as the present non-human animal in some cases);

2. The knockout non-human animal according to the above item 1, wherein the disruption leads to an insufficient expression of the PHF3 gene;

3. The knockout non-human animal according to the above item 1 or 2, wherein the disruption leads to the development of atopic dermatitis;

4. The knockout non-human animal according to any one of the above items 1 to 3, wherein the disruption prevents transcription of a full-length mRNA from the allele of the PHF3 gene;

5. The knockout non-human animal according to any one of the above items 1 to 4, wherein the disruption prevents transcription from exon 3 and the downstream region thereof in the allele of the PHF3 gene;

6. The knockout non-human animal according to any one of the above items 1 to 5, wherein the disruption comprises insertion of a gene cassette;

7. The knockout non-human animal according to the above item 6, wherein the gene cassette comprises a nucleotide sequence encoding a selectable marker;

8. The knockout non-human animal according to the above item 6, wherein the gene cassette comprises a nucleotide sequence encoding a selectable marker and a transcription termination signal;

9. The knockout non-human animal according to any one of the above items 1 to 8, wherein the disruption is a homozygous disruption;

10. The knockout non-human animal according to any one of the above items 1 to 9, wherein the non-human animal is a mammal;

11. The knockout non-human animal according to any one of the above items 1 to 9, wherein the non-human animal is a rodent;

12. The knockout non-human animal according to any one of the above items 1 to 9, wherein the non-human animal is a mouse;

13. The knockout non-human animal according to the above item 12, wherein the PHF3 gene comprises a nucleotide sequence encoding the amino acid sequence of SEQ ID NO: 1;

14. The knockout non-human animal according to any one of the above items 1 to 13, wherein the disruption leads to representation of one or more properties selected from the group consisting of the following (a) to (d):
(a) high-frequency formation of scabs on the skin as compared with the wild-type non-human animal;
(b) high-frequency cell infiltration into the dermis layer of the skin as compared with the wild-type non-human animal;
(c) a high level of total immunoglobulin E in the blood as compared with the wild-type non-human animal; and
(d) a variation in the expression amount of an atopic dermatitis-associated factor as compared with the wild-type non-human animal;

15. A tissue or cell isolated from the knockout non-human animal according to any one of above items 1 to 14;

16. The tissue or cell according to the above item 15, which is a T cell, a B cell or a leukocyte-derived cell;

17. A cultured animal cell whose genome comprises a disruption in at least one allele of the PHF3 gene;

18. The cultured animal cell according to the above item 17, wherein the disruption leads to an insufficient expression of the PHF3 gene;

19. The cultured animal cell according to the above item 17 or 18, which is totipotent;

20. The cultured animal cell according to any one of the above items 17 to 19, wherein the PHF3 gene comprises a nucleotide sequence encoding the amino acid sequence of SEQ ID NO: 1;

21. The cultured animal cell according to the above item 17 or 18, wherein the PHF3 gene comprises a nucleotide sequence encoding the amino acid sequence of SEQ ID NO: 7;

22. An animal model for a disease with the development of atopic dermatitis, comprising the non-human animal according to any one of the above items 1 to 14;

23. A method for analyzing a state of a disease associated with the PHF3 protein, which comprises performing on the non-human animal according to any one of the above items 1 to 14 during the period from the embryonic stage to death: observation of growth and differentiation, development, and living behavior; a histopathological test; or a biochemical test;

24. The analyzing method according to the above item 23, wherein the disease associated with the PHF3 protein is a disease with the development of atopic dermatitis;

25. A method of assessing an ability to control atopic dermatitis, which comprises:

(1) the first step of contacting the non-human animal according to any one of the above items 1 to 14 or a part thereof with a test substance,

(2) the second step of measuring an expression amount of an atopic dermatitis-associated factor in the non-human animal or a part thereof which has been contacted with the test substance in the first step, or an index value correlated with the expression amount, and then comparing the measured value with a control, and

(3) the third step of assessing an ability of the test substance to control atopic dermatitis on the basis of the comparison result of (2);

26. The assessing method according to the above item 25, wherein the ability to control atopic dermatitis is an ability to control the expression of an atopic dermatitis-associated factor;

27. The assessing method according to the above item 25 or 26, wherein the expression amount of an atopic dermatitis-associated factor is a RNA amount or a protein amount of an atopic dermatitis-associated factor;

28. The assessing method according to any one of the above items 25 to 27, wherein the atopic dermatitis associated factor is one or more factors selected from the group consisting of IFN-γ, IL-4, IL-5, IL-13, IL-18 and immunoglobulin E;

29. The assessing method according to the above item 28, wherein the atopic dermatitis-associated factor is immunoglobulin E;

30. The assessing method according to any one of the above items 25 to 28, wherein the index value correlated with the expression amount of an atopic dermatitis-associated factor is an amount of formed scabs;

31. The assessing method according to any one of the above items 25 to 28, wherein the index value correlated with the expression amount of an atopic dermatitis-associated factor is a cell infiltration amount into the dermis layer of the skin;

32. The assessing method according to any one of the above items 25 to 28, wherein the index value correlated with the expression amount of an atopic dermatitis-associated factor is the total immunoglobulin E level in the blood;

33. A method of screening an atopoic dermatitis controlling substance, which comprises selecting a test substance having an ability to control atopic dermatitis on the basis of an ability to control atopic dermatitis assessed by the assessing method according to any one of the above items 25 to 32;

34. A remedy or prophylactic for a disease with the development of atopic dermatitis, which comprises a substance having an ability to control atopic dermatitis obtained by the screening method according to the above item 33 as an active ingredient;

35. A recombinant vector for producing the non-human animal according to any one of the above items 1 to 14 or the cell according to any one of the above items 17 to 21, wherein the vector carries a gene cassette comprising a selectable marker gene from which a promoter region has been removed, and wherein the vector further comprises a nucleotide sequence homologous to a part of the nucleotide sequence of the PHF3 gene;

36. The recombinant vector according to the above item 35, wherein the gene cassette is a gene cassette in which mouse EN2-derived SA (splicing acceptor), encephalomyocarditis Virus-derived IRES (internal ribosome entry site), βgeo in which β-galactosidase and a neomycin resistant gene are fused, and SV40-derived pA (polyadenylation signal) are connected;

37. Use of a substance selected from:

(1) the PHF3 protein or a partial region thereof;

(2) a polynucleotide comprising a nucleotide sequence encoding the amino acid sequence of the protein or a partial region thereof described in (1), or a nucleotide sequence complementary to the nucleotide sequence;

(3) a DNA encoding a RNA having a ribozyme activity which specifically cleaves a nucleotide sequence encoding the amino acid sequence of the protein or a partial region thereof described in (1); and

(4) a DNA encoding a RNA which suppresses the expression of the PHF3 gene on the basis of RNAi effect when expressed in a cell, for protein therapy or gene therapy of an allergy disease;

38. The use according to the above item 37, wherein the PHF3 protein has an activity for ameliorating allergy disease symptoms and comprises any one of the following amino acid sequences:

<Amino Acid Sequence>

(a) the amino acid sequence of SEQ ID NO: 1 or 7;

(b) the amino acid sequence of SEQ ID NO: 1 or 7 in which one or more amino acids are deleted, added or substituted;

(c) an amino acid sequence with 75% or more sequence identity to the amino acid sequence of SEQ ID NO: 1 or 7;

(d) the amino acid sequence encoded by the nucleotide sequence of SEQ ID NO: 2 or 8;

(e) an amino acid sequence encoded by a DNA comprising a nucleotide sequence with 80% or more sequence identity to the nucleotide sequence of SEQ ID NO: 2 or 8; and

(f) an amino acid sequence encoded by a DNA which hybridizes with a DNA comprising a nucleotide sequence complementary to the nucleotide sequence of SEQ ID NO: 2 or 8 under stringent conditions;

39. An antibody immunologically specific for a protein comprising the amino acid sequence of SEQ ID NO: 1 or 7 or a partial sequence thereof;

40. Use of a substance selected from:

(1) the PHF3 protein or a partial region thereof;

(2) a polynucleotide comprising a nucleotide sequence encoding the amino acid sequence of the protein or a partial region thereof described in (1), or a nucleotide sequence complementary to the nucleotide sequence; and

(3) a gene of the PHF3 protein, for diagnosing atopic dermatitis;

41. The use according to the above item 40, wherein the PHF3 protein has an activity for ameliorating allergy disease symptoms and comprises any one of the following amino acid sequences:

<Amino Acid Sequence>

(a) the amino acid sequence of SEQ ID NO: 1 or 7;

(b) the amino acid sequence of SEQ ID NO: 1 or 7 in which one or more amino acids are deleted, added or substituted;

(c) an amino acid sequence with 75% or more sequence identity to the amino acid sequence of SEQ ID NO: 1 or 7;

(d) the amino acid sequence encoded by the nucleotide sequence of SEQ ID NO: 2 or 8;

(e) an amino acid sequence encoded by a DNA comprising a nucleotide sequence with 80% or more sequence identity to the nucleotide sequence of SEQ ID NO: 2 or 8; and

(f) an amino acid sequence encoded by a DNA which hybridizes with a DNA comprising a nucleotide sequence complementary to the nucleotide sequence of SEQ ID NO: 2 or 8 under stringent conditions; and the like.

### BRIEF DESCRIPTION OF DRAWINGS

FIG. 1 shows results of decoding of the nucleotide sequence of a trapped gene by a 5′ RACE method, using an ES cell used for producing a PHF3 gene knockout mouse. The decoded nucleotide sequence corresponded to the first exon and the second exon of the PHF3 gene.

FIG. 2 is a photograph of the appearance of a mouse with a homozygous knockout PHF3 gene. Hair loss and scab formation were observed over a wide region of the back.

FIG. 3 shows a stained image of tissues (abdominal) such as a skin and a subcutaneous muscle by HE-staining. (a) is a stained image of a thin section of a skin tissue from a normal mouse, (b) is a stained image of a thin section of a subcutaneous muscle tissue from a normal mouse, (c) is a stained image of a thin section of a skin tissue from the present knockout mouse, and (d) is a stained image of a thin section of a subcutaneous muscle tissue from the present knockout mouse. In the case of the present knockout mouse, proliferation of prickle cells, formation of scabs, and cell infiltration into the skin dermis layer were observed and, additionally, cell infiltration into a subcutaneous tissue and atrophy of cutaneous muscle were observed.

FIG. 4 shows results of measurement of the total immunoglobulin E levels in sera of PHF3 gene knockout mice (PHF3 homo KO) and wild-type mice (WK). In the case of the PHF3 gene homo-knockout mice, measured values of respective individuals are shown as bar graphs. In the case of the wide-type mice, the average of measured values of four individuals is shown as a bar graph, and the standard deviation is represented by an error line.

FIG. 5 shows results of the expression amount of PHF3 mRNA investigated in various human tissues. The upper column shows results of amplification of the PHF3 gene by a PCR method. The lower column shows results of amplification of the β actin gene by a PCR method. Of leukocyte samples, the “resting” shows cDNAs derived from hemocytes which were subjected to only a treatment of separation from blood, and the “activated” shows cDNAs derived from a mononuclear cell which was treated with concanavalin A (ConA) and pokeweed mitogen (PWM), a helper T cell which was treated with ConA, a killer T cell which was treated with phytohemagglutinin, and a B cell which was treated with PWM.

FIG. 6 shows analysis results of expression of the PHF3 gene in the lymph nodes of PHF3 homo-knockout mice and wide-type mice by a reverse transcription PCR method.

### BEST MODE FOR CARRYING OUT THE INVENTION

The present invention will be explained in detail below.

The present invention provides a knockout non-human animal whose genome comprises a disruption in at least one allele of the PHF3 gene. Herein, the “disruption in an allele” means that the allele(s) of an endogenous gene such as the PHF3 gene is artificially altered so that the expression of at least a part of a protein encoded by the endogenous gene is lost or reduced. Examples of such disruption include a heterozygous disruption in which one of alleles of an endogenous gene is disrupted, and a homozygous disruption in which both of the alleles of an endogenous gene are disrupted.

The knockout non-human animal in the present invention means a transgenic non-human animal in which all or one of alleles at a locus present in cells including germ line cells in the living body of a non-human animal is disrupted (e.g. disrupted by recombination), so that the original function of the gene is not exerted. In the case of the knockout non-human animal provided according to the present invention, all or one of alleles of the PHF3 gene at a locus on a chromosome in a cell constituting a non-human animal living body is disrupted (e.g. disrupted by recombination), so that the active PHF3 protein is not expressed. In such knockout non-human animal, artificial alteration of allele(s) may result in inhibition of the expression ability of the PHF3 gene or substantial loss or reduction of the activity of the PHF3 protein encoded by the allele(s).

The PHF3 gene in the present invention is a region on a chromosome of a non-human animal which corresponds to a mRNA having a nucleotide sequence encoding the amino acid sequence of the PHF3 protein and may contain introns, and it is not limited to specific structure, for example, a specific nucleotide sequence or specific intron-exon structure.

Examples of the PHF3 protein in the present invention (hereinafter, referred to as the present protein in some cases) include (a) a protein comprising the amino acid sequence of SEQ ID NO: 1, (b) a protein comprising the amino acid sequence of SEQ ID NO: 1 in which one or more amino acids are deleted, added or substituted, and having an activity for ameliorating allergy disease symptoms, (c) a protein comprising an amino acid sequence with 75% or more sequence identity to the amino acid sequence of SEQ ID NO: 1, and having an activity for ameliorating allergy disease symptoms, (d) a protein comprising the amino acid sequence encoded by the nucleotide sequence of SEQ ID NO: 2, (e) a protein comprising an amino acid sequence encoded by a DNA comprising a nucleotide sequence with 80% or more sequence identity to the nucleotide sequence of SEQ ID NO: 2, and having an activity for ameliorating allergy disease symptoms, and (f) a protein comprising an amino acid sequence encoded by a DNA which hybridizes with a DNA comprising a nucleotide sequence complementary to the nucleotide sequence of SEQ ID NO: 2 under stringent conditions, and having an activity for ameliorating allergy disease symptoms.

Herein, the “deletion, addition or substitution of amino acids” described in the (b), and the “amino acid sequence with 75% or more sequence identity” and the “nucleotide sequence with 80% or more sequence identity” described in the (c) and the (e) include naturally occurring mutations due to processing which a protein comprising the amino acid sequence of SEQ ID NO: 1 undergoes in a cell, a spices difference or an individual difference between organisms from which the protein is derived, a difference between tissues from which the protein is derived or the like, artificial amino acid mutations, and the like.

An example of an artificial procedure for the “deletion, addition or substitution of amino acid” described in the (b) (hereinafter, collectively referred to as “alteration of amino acid”) comprises introducing a mutation into a DNA encoding the amino acid sequence of SEQ ID NO: 1 by a conventional site-specific mutagenesis method and then expressing said DNA by a conventional method. Herein, examples of the site-specific mutagenesis method include a method utilizing amber mutation (gapped duplex method, Nucleic Acids Res., 12, 9441-9456(1986)), a PCR method using primers for introducing a mutation, and the like.

The number of amino acids to be altered as described above is at least one residue, specifically 1 or a few, or more. The number of such alteration may be in such a range that an activity for ameliorating allergy disease symptoms can be found in the present protein.

Among the aforementioned deletion, addition and substitution, alteration involving substitution of amino acid is particularly preferred. More preferably, the substitution is substitution with amino acid whose properties such as hydrophobicity, charge, pK, a steric structural characteristic and the like are similar. Examples of such substitution include substitution within the groups of i) glycine, alanine; ii) valine, isoleucine, leucine; iii) aspartic acid, glutamic acid, asparagine, glutamine, iv) serine, threonine; v) lysine, arginine, histidine; vi) phenylalanine, tyrosine.

The “sequence identity” in the present invention refers to identity and homology between two nucleic acid or two protein sequences. The “sequence identity” is determined by comparing two sequences aligned in the optimal state, over the region of a sequence to be compared. Herein, a nucleic acid or protein to be compared may have addition or deletion (e.g. gap etc.) in the optimal alignment of two sequences. Such sequence identity can be calculated by producing alignment utilizing the ClustalW algorism (Nucleic Acid Res., 22(22):4673-4680(1994)), for example, using Vector NTI. The sequence identity is determined using sequence analysis software, specifically, Vector NTI or GENETYX-MAC, or an analysis tool provided in public database. The public database is generally available, for example, in a website (http://www.ddbj.nig.ac.jp) of DNA Data Bank of Japan [an international DNA data bank operated in Center for International Biology and DNA Data Bank of Japan; CIB/DDBJ)] or the like.

The sequence identity in the present invention is 75% or more, preferably 85% or more, more preferably 90% or more at the amino acid level. At the nucleic acid level, the sequence identity is 80% or more, preferably 90% or more, more preferably 95% or more.

Regarding the “hybridizes under stringent conditions” described in the (f), the hybridization can be performed according to a conventional method described in, for example, Sambrook J., Frisch E. F., Maniatis, T., Molecular Cloning 2^{nd} edition, Cold Spring Harbor Laboratory Press or the like. An example of the “stringent conditions” is hybridization at 45° C. in a solution containing 6×SSC (a solution containing 1.5 M NaCl and 0.15 M trisodium citrate is defined as 10×SSC) and 50% formamide, followed by washing with 2×SSC at 50° C. (Molecular Biology, John Wiley & Sons, N.Y. (1989), 6.3.1-6.3.6). The salt concentration in a washing step can be selected, for example, from the range of 2×SSC at 50° C. (low stringent condition) to 0.2×SSC at 50° C. (high stringent condition). The temperature in a washing step can be selected, for example, from the range of room temperature (low stringent condition) to 65° C. (high stringent condition). In addition, both the salt concentration and the temperature may be changed.

More specific examples of the PHF3 gene in present invention include (I) a gene comprising a nucleotide sequence encoding the amino acid sequence of SEQ ID NO: 1 or 7, (II) a gene comprising the nucleotide sequence of SEQ ID NO: 2 or 8, and the like, and naturally occurring allele gene polymorphism of these genes is also included. These genes can be obtained according to a conventional genetic engineering method (e.g. a method described in Sambrook J., Frisch E. F., Maniatis T., Molecular Cloning 2^{nd} edition, Cold Spring Harbor Laboratory press etc.).

The nucleotide sequence of SEQ ID NO: 2 or 8 is a nucleotide sequence of a cDNA corresponding to a mRNA comprising a nucleotide sequence encoding the amino acid sequence of SEQ ID NO: 1 or 7 (i.e. a nucleotide sequence of a mRNA comprising a nucleotide sequence encoding the amino acid sequence of SEQ ID NO: 1 or 7 in which U is substituted with T), which comprises a nucleotide sequence of an untranslated region at the 5′-terminus and the 3′-terminus. The PHF3 genome gene contains a nucleotide sequence of a cDNA such as the nucleotide sequence of SEQ ID NO: 2, and has structure in which said nucleotide sequence is interrupted by introns, that is, intron-exon structure.

In the case of the nucleotide sequence of SEQ ID NO: 2, a more specific example of the intron-exon structure contains introns at positions between nucleotide numbers 312 and 313 (between exons 1 and 2), between nucleotide numbers 582 and 583 (between exons 2 and 3), between nucleotide numbers 744 and 745 (between exons 3 and 4), between nucleotide numbers 2461 and 2462 (between exons 4 and 5), between nucleotide numbers 2771 and 2772 (between exons 5 and 6), between nucleotide numbers 2955 and 2956, (between exons 6 and 7), between nucleotide numbers 3100 and 3101 (between exons 7 and 8), between nucleotide numbers 3257 and 3258 (between exons 8 and 9), between nucleotide numbers 3374 and 3375 (between exons 9 and 10), between nucleotide numbers 3506 and 3507 (between exons 10 and 11), between nucleotide numbers 3642 and 3643 (between exons 11 and 12), between nucleotide numbers 3838 and 3839 (between exons 12 and 13), between nucleotide numbers 3984 and 3985 (between exons 13 and 14), between nucleotide numbers 4076 and 4077 (between exons 14 and 15), and between nucleotide numbers 4272 and 4273 ((between exons 15 and 16) on the nucleotide sequence of SEQ ID No:2.

Specific examples of the PHF3 gene include the mouse PHF3 gene (SEQ ID NO: 2: GenBank accession No. XM_{—}129836, the corresponding mouse PHF3 protein SEQ ID No: 1: GenBank accession No. XP_{—}129836) and the human PHF3 gene (SEQ ID NO: 8: GenBank accession No. NM_{—}015153, the corresponding human PHF3 protein SEQ ID NO: 7: GenBank accession No. NP_{—}055968). However, the source of the PHF3 gene is not limited to a mouse and a human. Examples of the PHF3 gene also include orthologues of the PHF3 gene derived from a mouse or a human. The sequence identity between the mouse PHF3 gene and the human PHF3 gene is 82.0% at the nucleic acid level and 77.5% at the amino acid level.

Examples of the “activity for ameliorating allergy disease symptoms” as used herein include activities for ameliorating formation of scabs, cell infiltration into the skin dermis layer and a high level of total immunoglobulin E in the blood which are symptoms characteristic of atopic dermatitis. Other examples of the “activity for ameliorating allergy disease symptoms” include activities for ameliorating an abnormal level of a cytokine, an abnormal level of histamine, and the like.

The non-human animal in the present invention is an animal comprising the PHF3 gene other than a human. Examples of the non-human animal include non-human mammals (e.g. rabbit, dog, cat, guinea pig, hamster, mouse, rat, sheep, gout, pig, horse, bovine, monkey etc.). Inter allia, Robentia mammals such as a mouse, a rat, a guinea pig and the like is preferred, and a mouse and a rat are particularly preferred.

A part of the non-human animal in the present invention is not particularly limited as long as it is a tissue or a cell derived from the non-human animal. Examples of a part of the non-human animal include parts of the body including skin tissues such as a skin and a subcutaneous muscle, various fat tissues such as a peritesticular fat tissue, a retroperitoneal fat tissue, a mesenteric fat tissue, a subcutaneous fat tissue and a brown fat tissue, and other tissues such as heart, lung, kidney, gallbladder, liver, pancreas, spleen, intestine, orchis (testis), ovary, uterus, placenta, muscle, blood vessel, brain, marrow, thyroid gland, thymus, mammary gland and lymph node. In addition, body fluid such as blood, lymph fluid and urine derived from the non-human animal is also included in a part of the non-human in the present invention.

Further, examples of a part of the non-human animal include not only cultured cells obtained by isolating and culturing cells contained in the aforementioned tissues, organs or body fluid (including collected first-generation primary cells and cells established from the primary cells) and extracts, but also various organs at the developmental stage in an embryo at the embryonic stage, cultures of cells associated with the organs, and ES cells regardless of the presence or absence of their differentiation ability and proliferation ability.

Specific examples of such a tissue or cell derived from the non-human animal include cell strains derived from T cells, B cells and leukocytes and the like.

The first aspect of the present invention relates to an individual of a knockout non-human animal, a progeny animal thereof, or a part thereof.

The present non-human animal is provided, for example, as a heterozygote or a homozygote in which both or one of alleles of the diploid PHF3 gene on a chromosome in somatic cells is replaced with a function-deficient mutant or function-reduced mutant of the PHF3 gene, or all or one of alleles of the diploid genome gene is disrupted (e.g. disrupted by recombination) so that its function is deleted or reduced.

The present non-human animal can be produced according to a conventional method such as a known target gene recombination method (gene targeting method: e.g. Masahiro Muramatsu, Masa Yamamoto ed., “Experimental Medicine Separate Volume, New Edition, Genetic Engineering Handbook 3^{rd} edition” (published by Yodosha in 1999), p. 239-256, Science 244:1288-1292,1989). For example, both or one of alleles of the targeted diploid genome gene (i.e. the genome gene of the PHF3 protein) on a chromosome in ES (embryonic stem) cells of a non-human animal whose PHF3 gene is to be altered is disrupted or recombined, for example using a targeting vector carrying a selectable marker gene (i.e. a DNA comprising a nucleotide sequence encoding a selectable marker) from which a promoter region has been removed (selectable marker gene promoterless recombinant vector), by a homologous recombination method (i.e. promoter trap method) by which a part of the targeted gene on a chromosome is replaced with the targeting vector. After the ES cell is mixed with the blastocyst of a parent animal, embryonic differentiation is allowed to continue until a baby is born, and thereby a chimeric primitive mouse is produced as an offspring. In this method, ES cells and the like are used as totipotent cells. As the totipotent cells, ES cells and the like of a mouse (Nature 292:154-156, 1981), a rat (Dev. Biol. 163(1): 288-292, 1994), a monkey (Proc. Natl. Acad. Sci. U.S.A. 92(17): 7844-7848, 1995) and a rabbit (Mol. Reprod. Dev. 45(4): 439-443, 1996) have been established. In addition, an EG (embryonic germ) cell of a pig has been established (Biol. Reprod 57(5): 1089-1095, 1997). Therefore, the present non-human animal is preferably produced with these animal species. In particular, a mouse is optimal because technique is ready for production of a knockout animal using a mouse.

A specific procedure for producing the present non-human animal will be explained below. First, a DNA of a genome gene encoding the PHF3 protein is isolated, and then subjected to gene manipulation in vitro to disrupt the genome gene so that the expression of the genome gene becomes insufficient. Examples of a method for such artificial alteration of the PHF3 gene include a method comprising deletion or substitution of a part or all of the nucleotide sequence of the gene by a genetic engineering procedure, a method comprising insertion of other genes or replacement with other genes, and the like. Such alteration results in a frame shift in an open reading frame of codons, insertion of a nucleotide sequence which terminates transcription of a gene (e.g. polyA addition signal etc.) into an intron part between exons, disruption of the function of a promoter or an exon, or the like, so that the expression of the gene can be prevented.

Specifically, for example, a mutated DNA is produced to replace the genome gene with a function-deficient mutant or a function-reduced mutant of the gene, or disrupt or recombine the genome gene so that its function is deleted or reduced.

A DNA of the genome gene is obtained, for example, by screening a genome DNA library of a non-human animal such as a mouse using an oligonucleotide probe synthesized based on a known cDNA nucleotide sequence of the mouse-derived PHF3 gene. Alternatively, a DNA of the objective genome gene can be also obtained by a PCR method using synthetic oligonucleotides corresponding to a part or both ends of the aforementioned cDNA as primers. When a non-human animal other than a mouse is a subject, a DNA of the gene derived from various non-human animals can be isolated by the aforementioned methods using oligonucleotides synthesized based on the nucleotide sequence of the aforementioned cDNA as probes or primers. In addition, based on the nucleotide sequence of the aforementioned cDNA, the structure of the genome gene is analyzed in detail to elucidate intron-exon structure in the genome gene and the nucleotide sequences of introns.

Based on the elucidated information, for example, a DNA containing a part of the genome gene can be altered by replacement, deletion and/or addition so that at least a part of a nucleotide sequence in a protein-encoding region of the gene is changed, to produce a mutated DNA for replacing the genome gene with a function-deficient mutant or a function-reduced mutant of the gene, or disrupting or recombining the genome gene so that its function is deleted or reduced.

The mutated DNA thus prepared by altering a part of a DNA of the PHF3 gene is used to prepare a targeting vector for introducing a mutation into the PHF3 gene in a totipotent cell such as an ES cell according to a known method (e.g. Science 244: 1288-1292, 1989). Herein, the “targeting vector” is a DNA molecule for disrupting or recombining an objective gene by, for example, homologous recombination, and usually contains a nucleotide sequence of a selectable marker gene for facilitating selection of a totipotent cell such as ES cell in which recombination has occurred. Examples of a positive selectable marker gene include a neomycin resistant gene, a β-galactosidase gene and the like. Examples of a negative selectable marker gene include a herpes simplex virus thymidine kinase gene, a diphtheria toxin A fragment gene and the like. Upon construction of the targeting vector, a commercially available plasmid vector for constructing a targeting vector may be utilized.

An example of a method for preparing a targeting vector which is preferably used in the present invention comprises using a selectable marker gene promoterless recombinant vector which carries a gene cassette containing a selectable marker gene from which a promoter region has been removed and which has a nucleotide sequence homologous to a part of a DNA of the PHF3 gene, replacing at least a part of a DNA within a protein-coding region of the PHF3 genome gene with a DNA which is a part or all of the vector and contains the gene cassette to prepare a selectable marker gene promoterless recombinant vector which carries a gene cassette containing a selectable marker gene from which a promoter region has been removed, and which has a nucleotide sequence homologous to a part of a DNA of the PHF3 gene on the both sides of the gene cassette. Another example of a method for preparing a targeting vector comprises using a selectable marker gene promoterless recombinant vector which carries a gene cassette containing a selectable marker gene from which a promoter region has been removed and which has a nucleotide sequence homologous to a part of a DNA of the PHF3 gene, inserting a DNA which is a part or all of the vector and contains the gene cassette into at least a part of a DNA within a protein-coding region of the PHF3 genome gene to prepare a selectable marker gene promoterless recombinant vector which carries a gene cassette containing a selectable marker gene from which a promoter region has been removed, and which has a nucleotide sequence homologous to a part of DNA of the PHF3 gene on the both sides of the gene cassette. In these cases, replacement or insertion may be performed in a plasmid vector for constructing a targeting vector into which the DNA to be replaced or inserted as described above has been incorporated in advance, or the DNA which has been already replaced or inserted as described above may be incorporated into a plasmid vector for constructing a targeting vector. When the PHF3 protein consists of the amino acid sequence of SEQ ID NO: 1, examples of the “at least a part of a DNA within a protein-coding region of the PHF3 genome gene” include nucleotides within the exon 3 region (herein, the “nucleotides within the exon 3 region” may be, for example, all nucleotides within the exon 3 region, or a part of nucleotides within the exon 3 region), and more preferably all nucleotides within the exon 3 region; provided that, in selection of exon herein, it is not preferable that the region of 2+3n (n is the number of amino acids within a region from the front position of exon 3) nucleotides from the front position of exon 3 is selected because in this case, a translation frame following exon 2 is consistent with a downstream gene of a protein-coding region of the PHF3 gene and thereby there is a possibility that a protein fused with the downstream region is produced.

Specifically, examples of the “gene cassette containing a selectable marker gene from which a promoter region has been removed” include a DNA (SA-IRES-βgeo-pH gene cassette) in which mouse EN2-derived SA (splicing accepter), encephalomyocarditis Virus-derived IRES (internal ribosome entry site), βgeo in which β-galactosidase and neomycin resistant genes are fused, and SV40-derived pA (polyadenylation signal) (Mountford P. et al., PNAS 91:4303-4307, 1994) are connected, and the like. A gene cassette containing a DNA comprising a nucleotide sequence encoding a selectable marker (i.e. a selectable marker gene from which a promoter region has been removed) is inserted, for example, between exon 2 and exon 3 of a genome gene encoding the PHF3 protein by homologous recombination utilizing the aforementioned targeting vector, so that it becomes impossible to synthesize a full length mRNA from the PHF3 gene and, consequently, the PHF3 gene can be disrupted. The gene cassette may contain a transcription termination signal (e.g. polyA addition signal) or the like, in addition to a DNA comprising a nucleotide sequence encoding a selectable marker. In addition, the gene cassette may have a part of a nucleotide sequence homologous to the DNA nucleotide sequence of the PHF3 genome gene in regions before and after a DNA comprising a nucleotide sequence encoding a selectable marker.

Herein, the “homologous nucleotide sequence” is not particularly limited as long as it is a nucleotide sequence which has-identity or homology to a nucleotide sequence of a DNA to be compared and which does not cause a trouble upon implementation of a homologous recombination method. However, examples thereof include a nucleotide sequence which does not differ by continuous 3 or more nucleotides from a nucleotide sequence of a DNA to be compared and has 90% or more sequence identity to a nucleotide sequence of a DNA to be compared, preferably a nucleotide sequence which has 95% or more sequence identity to a nucleotide sequence of a DNA to be compared, more preferably a nucleotide sequence which has 100% sequence identity to a nucleotide sequence of a DNA to be compared.

For the aforementioned targeting vector, it is important to contain a gene cassette containing a selectable marker gene (e.g. a resistant gene to a cell toxin such as G418 etc., for example, a neomycin resistant gene) from which a promoter region has been removed and have a nucleotide sequence homologous to a part of the PHF3 gene, and the other factors are not particularly limited as long as they do not cause a trouble upon implementation of a homologous recombination method. In the present invention, the targeting vector is referred to as a selectable marker gene promoterless recombinant vector in some cases, as described above and below.

Then, the targeting vector is introduced into a totipotent cell such as an ES cell derived from a non-human animal such as a mouse according to a known method. Examples of such a gene introduction method include a known electric pulse method, liposome method, calcium phosphate method and the like. When the homologous recombination efficiency of an introduced gene is taken into consideration, an electric pulse method into a totipotent cell such as an ES cell is preferable.

The DNA of a totipotent cell such as an ES cell into which a gene has been introduced as described above is extracted, and the extracted DNA is subjected to Southern blotting analysis, PCR analysis and the like. Based on the results, a cell in which the aimed homologous recombination occurs between the wild-type genome gene of the PHF3 protein present on a chromosome (specifically, for example, at least a part of a DNA in a protein-coding region of the genome gene) and a DNA which is a part of the DNA of the targeting vector and contains the gene cassette and thereby a mutation is introduced into the wild-type genome gene of the PHF3 protein on a chromosome is selected. The selected ES cell and the wild-type ES cell are subjected to PCR real time quantification (RT-PCR method: Absolute Quantification Real-time Analysis) or the like, and the results can be compared to investigate an increase or a decrease in the expression amount of the PHF3 gene.

In the aforementioned method, after homologous recombination occurs in a chromosome in a totipotent cell such as an ES cell, only when a selectable marker is expressed in the ES cell utilizing a promoter of the PHF3 gene, the property of the selectable marker such as resistance function to a cell toxin is conferred on the cell. When the PHF3 gene is disrupted, the appearance efficiency of a clone in which homologous recombination occurs at the objective position to disrupt the PHF3 gene is enhanced by using said method, so that the objective clone can be effectively obtained.

The thus obtained totipotent cell such as an ES cell comprising the mutated genome gene is injected into the blastocyst of a wild-type female non-human animal such as a wild-type female mouse in the pseudo pregnant state according to a conventional microinjection method or aggregation method. Then, the resulting chimeric embryo is transplanted into the uterus of a foster parent. After embryonic differentiation is continued, for example, after 21 days in the case where the non-human animal is a mouse, a candidate chimeric non-human animal is born as an offspring. After the born candidate chimeric non-human animal is reared by a foster parent, a chimeric non-human animal (FO) comprising a mutated genome gene of the PHF3 protein introduced into a germ line cell is selected.

The selection may be performed by observing a difference in their fur color (the body hair color of the born chimeric non-human animals). (In this case, selection of a chimeric non-human animal comprising a high proportion of a recombinant totipotent cell such as a recombinant ES cell into which the mutated genome gene introduced becomes easy, so that the efficiency of acquiring a chimeric non-human animal comprising the mutated genome gene introduced into a germ line cell can be enhanced). Alternatively, the selection may be performed by extracting a DNA from a part of the body (e.g. the tip of a tail) and then subjecting the DNA to Southern blotting analysis, PCR analysis or the like.

A progeny (F1) obtained by mating a chimeric non-human animal comprising a mutated genome gene of the PHF3 protein introduced into a germ line cell with the wild-type non-human animal which belongs to the same animal species, is further subjected to Southern blotting analysis, PCR analysis or the like using a DNA extracted from a part of the body (e.g. the tip of a tail) as a material, and thereby, a heterozygote in which a mutated genome gene of the PHF3 protein is introduced (a non-human animal in which one of alleles of the diploid PHF3 gene is disrupted) is identified. In addition, an increase or a decrease in the expression amount of the PHF3 protein gene can be investigated, for example, by performing PCR real time quantification analysis or Northern blotting analysis using a RNA extract from a part of skin tissues such as a skin and a subcutaneous muscle, various fat tissues such as a peritesticular fat tissue, a retroperitoneal fat tissue, a mesenteric fat tissue, a subcutaneous fat tissue and a brown fat tissue, and other tissues such as heart, lung, kidney, gallbladder, liver, pancreas, spleen, intestine, orchis (testis), ovary, uterus, placenta, muscle, blood vessel, brain, marrow, thyroid gland, thymus, mammary gland and lymph node, or from an embryo at the embryonic stage as a material, and then comparing results. Further, increase and decrease in the amount of the PHF3 protein can be investigated by performing an ELISA (Enzyme Linked Immunosorbent Assay) method using the aforementioned body parts (mashed entity, section etc.) or body fluid (blood, urine etc.) as a material, and then comparing results.

The produced heterozygote comprising a mutated genome gene of the PHF3 protein stably possesses the mutated genome gene of the PHF3 protein in all of germ cells and somatic cells, and can effectively transmit the mutation to a progeny animal by mating or the like. Therefore, for progeny animals after F1, identification of heterozygotes, and investigation of increase and decrease in the expression amount of the gene and the amount of the protein which is the expression product of the gene can be performed in the same way as described above.

Generally, when a disrupted gene does not influence the development or differentiation of an embryonic individual and does not lead to embryonic death, in individuals after F1, genotypes of a homozygote (a non-human animal comprising the PHF3 gene whose all alleles in the diploid are disrupted or recombined), a heterozygote and the wild-type are produced at a probability in accordance with Mendel's laws. To the contrary, when a gene comprising a nucleotide sequence encoding the amino acid sequence of a protein essential for embryonic development or differentiation in the used non-human animal is disrupted, the survival and birth of a heterozygous or homozygous non-human animal are difficult, leading to embryonic death. This can be also inferred by comparing the actual birth rate and the predicted probability of appearance even when a heterozygote is obtained.

Generally, it is desirable that an animal used for experiment is an animal established as a line, that is, a genetically homogeneous animal. The genetic background of such homogeneous animal established as a line has been investigated in detail. In study using such experimental animals having the known genetic background, a treatment in the experimental animals produces a single effect, which can be easily identified. On the other hand, in a hybrid animal, various genes are present in the hetero state. Thus, a treatment in hybrid animals produces an effect as the sum of respective responses of individuals having respective genetic backgrounds to the treatment, which is extremely complicated. Therefore, it is difficult to determine whether an effect produced by a certain treatment is due to heterogeneity of the treatment or heterogeneity of the genetic background.

In the thus produced non-human animal or a progeny animal thereof (i.e. an individual of a knockout non-human animal or a progeny animal thereof), various blood biochemical tests, hemocyte tests and urine tests, various histopathological analyses, measurement of the total immunoglobulin E level in blood, and the like are performed, and thereby a difference in physiological action between the present knockout non-human animal and the wild-type non human animal (a non-human animal whose PHF3 gene is not disrupted) is investigated. Measurement of the total immunoglobulin E level in blood is a representative test which can be an index for assessment of atopic dermatitis.

As a result, as shown in Examples herein, in the present knockout non-human animal (e.g. 8 week-old mouse), formation of scabs and cell infiltration into the skin dermis layer are observed frequently as compared with the wild-type non-human animal. Further, the present knockout non-human animal (e.g. 43 week-old mouse) exhibits a finding of an extremely high level of the total immunoglobulin E in blood as compared with the wild-type non-human animal. From these findings, it can be confirmed that the present knockout non-human animal has the characteristic that exhibits abnormality in the immune system function and develops atopic dermatitis.

Based on the above results, the present knockout non-human animal or a part thereof and the wild-type non-human animal or a part thereof can be compared to know a difference in the development state of a disease with the development of atopic dermatitis.

Further, observation of growth and differentiation, development, and living behavior, a histopathological test or a biochemical test are performed on the present non-human animal or a part thereof during the period from the embryonic stage to death, and thereby the state of a disease associated with the PHF3 protein can be analyzed.

The present invention further provides a cultured animal cell comprising a disruption in at least one allele of the genome gene of the PHF3 protein comprising the amino acid sequence of SEQ ID NO: 7; more specifically, a primary cultured or established animal cell comprising a replacement of both or one of alleles of the diploid genome gene of a protein comprising the amino acid sequence of SEQ ID NO: 7 with a function-deficient mutant or a function-reduced mutant of the gene, or such a disruption (e.g. disruption by recombination) in all or one of alleles of the diploid genome gene as to delete or reduce its function, and a primary cultured or established animal cell comprising a replacement of all or one of alleles of the diploid genome gene of a protein comprising the amino acid sequence encoded by the nucleotide sequence of SEQ ID NO: 8 with a function-deficient mutant or a function-reduced mutant of the gene, or such a disruption (e.g. disruption by recombination) in all or one of alleles of the diploid genome gene as to delete or reduce its function.

The second aspect of the present invention relates to an animal model for a disease with the development of atopic dermatitis, comprising the present knockout non-human animal.

The present knockout non-human animal has a significantly reduced expression amount of the PHF3 protein as compared with the wild-type, and can express a disease associated with the protein. That is, as described above, the present knockout non-human animal specifically exhibits the following findings:
(a) formation of scabs on the skin
(b) cell infiltration into the skin dermis layer
(c) a high level of total immunoglobulin E in the blood
(d) variation in the expression amount of an atopic dermatitis-associated factor.

Therefore, the present knockout non-human animal can be used as an animal model for a disease with the development of atopic dermatitis, for 1) analysis of the state of a disease with the development of atopic dermatitis and the like, or 2) assessment of the atopic dermatitis treating effect or the atopic dermatitis development preventing effect of drugs, foods, drug candidates or food candidates.

Herein, “analysis of the state of a disease” means analysis of every change in a living body caused by a disease with the development of atopic dermatitis.

That is, the analysis of the state of a disease can be accomplished by performing observation of growth and differentiation, development, and living behavior, a histopathological test or a biochemical test during the period from the embryonic stage to death while comparing the present knockout non-human animal and the wild-type human animal. In the analysis of the disease state, the observation and tests may be performed according to a method usually used by a person skilled in the art, and “Transgenic Animal” edited by Kenichi Yamamura et al. (Kyoritsu Shuppan Co., Ltd.) and the like can be referenced.

For example, the total RNA extracted from a tissue of the present knockout non-human animal can be analyzed by PCR real time quantification analysis, Northern blotting analysis or the like, and then compared with analytical values of the wild-type non-human animal to identify a gene whose expression is induced or suppressed in the tissue. Based on the results, a marker gene whose expression is changed in a disease with the development of atopic dermatitis can be searched and, consequently, this can be used as an index for elucidating a causative factor of a disease.

Actually, as shown in Examples of the present invention, the present knockout non-human animal exhibits a change in the expression amount of an atopic dermatitis-associated factor. That is, the present knockout non-human animal exhibits an elevated expression of immunoglobulin E in blood, and thus the elevated expression of immunoglobulin E in blood can be used as an index to determine the presence or absence of a disease in a model animal for a disease with the development of atopic dermatitis.

A method for assessing an atopic dermatitis treating effect or an atopic dermatitis development preventing effect of drugs, foods, drug candidates or food candidates will be explained in detail below.

The third aspect of the present invention relates to a method for assessing an atopic dermatitis treating effect or the atopic dermatitis development preventing effect of a test substance using the present knockout non-human animal or a part thereof.

The produced non-human animal or a progeny animal or a part thereof (i.e. an individual of the knockout non-human animal, or a progeny animal thereof, or a part thereof) is useful in the field of development of drugs or foods for diseases associated with atopic dermatitis. In addition, the non-human animal or a progeny animal thereof, or a part thereof can be used for assessing atopic dermatitis controlling substances.

The assessment method of the present invention comprises:

(1) the first step of contacting the present non-human animal or a part thereof with a test substance,

(2) the second step of measuring an expression amount of an atopic dermatitis-associated factor in the non-human animal or a part thereof which has been contacted with the test substance, or an index value correlated with the expression amount, and then comparing the measured value with a control, and

(3) the third step of assessing an atopic dermatitis treating effect or an atopic dermatitis development preventing effect of the test substance on the basis of the comparison result of (2).

The atopic dermatitis controlling substance herein refers to a substance having an atopic dermatitis treating effect or an atopic dermatitis development preventing effect.

The “atopic dermatitis treating effect or atopic dermatitis development preventing effect” means a treating effect or a development preventing effect on atopic dermatitis based on activity of ameliorating symptoms characteristic of atopic dermatitis, for example, formation of scabs, cell infiltration into the skin dermis layer and a high level of total immunoglobulin E in blood.

Herein, the test substance is not particularly limited, and may be a nucleic acid, a peptide, a protein (including an antibody directed to the present protein), an organic compound, an inorganic compound or the like. Examples of the test substance include a cell extract, an expression product of a gene library, a synthetic low-molecular organic compound, a synthetic peptide, a synthetic nucleic acid, a natural compound and the like. Herein, examples of the “antibody directed to the present protein” include antibodies immunologically specific for a protein comprising the amino acid sequence of SEQ ID NO: 1 or 7, or a partial region thereof.

Herein, the “contacting the present knockout non-human animal or a part thereof with a test substance” means administrating a test substance to the non-human animal, or contacting a test substance with a part of the non-human animal, and can be performed by a method generally used by a person skilled in the art.

When the test substance is administered to the non-human animal, a method for administering is not particularly limited, and may be oral or parenteral administration. Examples of a parenteral administration method include intravenous administration, subcutaneous administration, intracutaneous administration, intraperitoneal administration (ip), rectal administration, transdermal administration (application) and the like.

The form of the test substance is not particularly limited, and can be used as a solid, a liquid, a mixture with a base, a suspension, a solution or the like. When the test substance is formulated into a suspension or a solution, water, a pH buffer, a methylcellulose solution, a physiological saline, an organic solvent aqueous solution (as an organic solvent, ethanol and dimethyl sulfoxide are usually used) or the like is used. Examples of the base include oils such as glycerin, squalane and the like, and these are mainly used for preparing a test substance for application.

Examples of a method for measuring the “expression amount of an atopic dermatitis-associated factor” include a method comprising measurement of the RNA amount of the factor and a method comprising measurement of the protein amount of the factor, and said method may be performed in the same manner as a method for measuring the expression amount of an atopic dermatitis-associated factor as described below or a method described in Examples.

Examples of the “index value correlated with the expression amount of an atopic dermatitis-associated factor” in the second step of the assessment method include:
(a) formation of scabs on the skin,
(b) cell infiltration into the skin dermis layer, and
(c) the total immunoglobulin E level in blood.
For the heterozygote of the present invention, an additional example of the index value is the expression amount of PHF3.

An example of a method for measuring formation of scabs on the skin comprises observation with naked eyes.

An example of a method for measuring cell infiltration into the skin dermis layer comprises fixing a section of the skin or bone marrow, HE-staining the section to prepare a specimen, and then observing the specimen.

As a method for measuring an atopic dermatitis-associated factor, a method using an ELISA kit well-known to a person skilled in the art, or the like can be used.

Besides, examples of a procedure for biochemically analyzing the present knockout non-human animal include a method well-known to a person skilled in the art, and a method described in “The Journal of Clinical Investigation, April 2002, vol. 109, no. 8 (authored by Robert V. Farese Jr. et al.)” or “Nature Genetics, vol. 25, May 2000 (authored by Robert V. Farese Jr. et al.)”.

Herein, the “atopic dermatitis-associated factor” is, for example, a factor involved in the activation process of a T cell by antigen stimulation, a factor involved in the activation process of a B cell by a T cell, a factor involved in inflammation, or the like, and examples include one or more factors selected from IFN-γ, IL-4, IL-5, IL-13, IL-18, Immunoglobulin E and the like. When the expression amount of the atopic dermatitis-associated factor is measured, RNA or a protein may be used as a subject to be measured. It is preferable that a test substance which reduces the amount of IFN-γ, IL-4, IL-5, IL-13, IL-18 or immunoglobulin E is selected as a candidate substance of a remedy for an allergy disease or a prophylactic for the development of an allergy disease.

Each of them will be described in detail below. 1) The Case Where RNA is Used as a Subject to be Measured

In the case where an RNA is utilized as a biological sample, in order to measure the expression amount of the atopic dermatitis-associated factor, the expression level of an IFN-γ gene, an IL-4 gene, an IL-5 gene, an IL-13 gene or an IL-18 gene per unit amount of the total RNA contained in the biological sample may be detected and then measured.

The animal species from which the atopic dermatitis-associated factor gene is derived is not particularly limited. Usually, the gene derived from the same species as the present non-human animal is used.

Examples of the IFN-γ gene include a mouse-derived IFN-γ gene (Genbank Accession No. NM_{—}008337), a human-derived IFN-γ gene (Genbank Accession No. NM_{—}000619) and the like.

Examples of the IL-4 gene include a mouse-derived IL-4 gene (Genbank Accession No. NM_{—}021283), a human-derived IL-4 gene (Genbank Accession No. NM_{—}000589) and the like.

Examples of the IL-5 gene include a mouse-derived IL-5 gene (Genbank Accession No. NM_{—}010558), a human-derived IL-5 gene (Genbank Accession No. NM_{—}000879) and the like.

Examples of the IL-13 gene include a mouse-derived IL-13 gene (Genbank Accession No. NM_{—}008355), a human-derived IL-13 gene a human-derived IL-13 gene (Genbank Accession No. NM_{—}002188) and the like.

Examples of the IL-18 gene include a mouse-derived IL-18 gene (Genbank Accession No. NM_{—}008360), a human-derived IL-18 gene (Genbank Accession No. NM_{—}001562) and the like.

In the present non-human animal, a progeny thereof or a part thereof, the presence or absence or the extent of expression of the atopic dermatitis-associated factor gene (or the gene product) can be measured by detecting the presence or absence or the extent of expression of the gene.

An increase or a decrease in the expression amount of the atopic dermatitis-associated factor gene or a trend in the expression amount of other genes associated therewith is investigated by performing PCR real time quantification analysis or Northern blotting analysis using an RNA extracted from each tissue of the present knockout non-human animal as a sample, and thereby the development state of a disease with the development of atopic dermatitis can be known. Consequently this investigation can be used as an index for elucidating a causative factor of the disease.

Specifically, a biological sample derived from the present non-human animal is contacted with a primer or probe derived from an atopic dermatitis-associated factor gene, and the amount of an RNA binding with the primer or probe can be measured by a known method such as a Northern blotting method, a RT-PCR method, a DNA chip analysis, an in situ hybridization analysis or the like. Examples of the primer or probe include a polynucleotide comprising at least 15 continuous nucleotides in the nucleotide sequence of the atopic dermatitis-associated factor gene, and/or a polynucleotide complementary to them.

Examples of a polynucleotide used as the primer include polynucleotides having a nucleotide length of usually 15 bp to 100 bp, preferably 15 bp to 50 bp, more preferably 15 bp to 35 bp. Examples of a polynucleotide used as the detection probe include polynucleotides having a nucleotide length of usually 15 bp to the full length, preferably 15 bp to 1 kb, more preferably 100 bp to 1 kb.

When a Northern blotting method is utilized, specifically, for example, the probe is labeled with a radioactive isotope element (32P, 33P etc.: RI) or a fluorescent substance, and the labeled probe is hybridized with an RNA derived from a living body tissue of a subject which has been transferred to a nylon membrane or the like according to a conventional method. A duplex formed between the primer (DNA or RNA) derived from an atopic dermatitis-associated factor and the total RNA derived from a living body sample is measured by detecting a signal derived from the label of the primer (RI or fluorescent substance) with a radiation detector (BAS-1800II, manufactured by Fuji Photo Film Co., Ltd.) or a fluorescent light detector. Alternatively, a measuring method which comprises labeling a probe DNA, hybridizing the probe with RNA derived from a biological sample and then detecting a signal derived from the label of the probe with a multibioimager STORM860 (manufactured by Amersham Pharmacia Biotech) using AlkPhos Direct Labelling and Detection System (manufactured by Amersham Pharmacia Biotech) according to the protocol may be used.

When a RT-PCR method is utilized, a method which comprises hybridizing the primer with an RNA derived from a biological sample, performing a PCR method according to a conventional method and then detecting an amplified double-stranded DNA can be exemplified. For detecting the amplified double-stranded DNA, a method which comprises detecting a produced labeled double-stranded DNA by performing the PCR using a primer which has been labeled with RI or a fluorescent substance in advance, a detecting method which comprises transferring a produced double-stranded DNA to a nylon membrane or the like according to a conventional method, and hybridizing a labeled disease marker as a probe with the nylon membrane or the like, or the like can be used. The produced labeled double-stranded DNA product can be measured with an Agilent 2100 bioanalyzer (manufactured by Yokokawa Analytical systems) or the like. Alternatively, a RT-PCR reaction solution is prepared with SYBR Green RT-PCR Regents (manufactured by Applied Biosystems) according to the protocol, and reacted with ABI PRIME 7700 Sequence Detection System (manufactured by Applied Biosystems), thereby, the reaction product may be detected.

When DNA chip analysis is utilized, a method which comprises preparing a DNA chip on which the primer or probe is stuck as a DNA probe (single-stranded or double-stranded), hybridizing the chip with a cRNA prepared from an RNA derived from a biological tissue according to a conventional method, and then binding a duplex formed between the DNA and the cRNA to a labeled probe prepared from the primer or probe to detect the RNA can be exemplified. As the DNA chip, a DNA chip which can detect or measure the gene expression level of an IFN-γ gene, an IL-4 gene, an IL-5 gene, an IL-13 gene, an IL-18 gene or an immunoglobulin E gene may be used.

2) The Case Where a Protein is Used as a Subject to be Measured

In the case where a protein is utilized as a biological sample, in order to measure the expression amount of the atopic dermatitis-associated factor, the expression level of IFN-γ, IL-4, IL-5, IL-13, IL-18 or immunoglobulin E per unit amount of the total protein contained in the biological sample may be detected and then measured.

The animal species from which the atopic dermatitis-associated factor (protein) is derived is not particularly limited. Usually, the protein derived from the same species as the present non-human animal is used.

Examples of the IFN-γ include mouse-derived IFN-γ (Genbank Accession No. NP_{—}0032363), human-derived IFN-γ (Genbank Accession No. NP_{—}000610) and the like.

Examples of the IL-4 include mouse-derived IL-4 (Genbank Accession No. NP_{—}067258), human-derived IL-4 (Genbank Accession No. NP_{—}000580) and the like.

Examples of the IL-5 include mouse-derived IL-5 (Genbank Accession No. NP_{—}034688), human-derived IL-5 (Genbank Accession No. NP_{—}000870) and the like.

Examples of the IL-13 include mouse-derived IL-13 (Genbank Accession No. NP_{—}032381), human-derived IL-13 (Genbank Accession No. NP_{—}002179) and the like.

Examples of the IL-18 include mouse-derived IL-18 (Genbank Accession No. NP_{—}032386), human-derived IL-18 (Genbank Accession No. NP_{—}001553) and the like.

Examples of immunoglobulin E include immunoglobulin E derived from human (Genbank Accession No. XP_{—}370382) and the like.

In the case where a solution containing a protein is utilized as a biological sample, for example, an atopic dermatitis-associated factor contained in the biological sample is reacted with an antibody capable of recognizing the atopic dermatitis-associated factor, and the amount of the atopic dermatitis-associated factor capable of binding to the antibody is detected and then measured.

The animal species from which an antibody capable of recognizing an atopic dermatitis-associated factor is derived is not particularly limited. Usually, an antibody obtained by using an atopic dermatitis associated factor derived from the same species as the present non-human animal as an antigen is used.

The antibody of the atopic dermatitis-associated factor is not particularly limited in its form, but may be a polyclonal antibody or a monoclonal antibody directed to the atopic dermatitis-associated factor as an immunological antigen. Further, an antibody having the antigen binding property to a polypeptide comprising continuous at least about 3 amino acids, usually about 8 amino acids, preferably about 15 amino acids of the amino acid sequence constituting the atopic dermatitis-associated factor may be used.

These antibodies can be produced according to a conventional method (Current protocols in Molecular Biology edit. Ausubel et al. (1987) Publish. John Wiley and Sons. Section 11.12-11.13). Specifically, in the case of a polyclonal antibody, a non-human animal such as a domestic rabbit is immunized using IFN-γ, IL-4, IL-5, IL-13, IL-18 or immunoglobulin E which has been expressed in Escherichia coli or the like and purified according to a conventional method, or using an oligopeptide comprising a partial amino acid sequence of any of the proteins synthesized according to a conventional method. An objective polyclonal antibody can be obtained from the serum of the immunized animal according to a conventional method. On the other hand, in the case of a monoclonal antibody, a non-human animal such as a mouse is immunized with IFN-γ, IL-4, IL-5, IL-13, IL-18 or immunoglobulin E which has been expressed in Escherichia coli or the like and purified according to a conventional method, or an oligopeptide comprising a partial amino acid sequence of these proteins. The spleen cells obtained from the immunized animal are fused to myeloma cells to prepare hybridoma cells, and then, an objective monoclonal antibody can be obtained from among the hybridoma cells (Current protocols in Molecular Biology edit. Ausubel et al. (1987) Publish. John Wiley and Sons. Section 11.4-11.11).

A protein used in production of an antibody can be obtained by performing DNA cloning based on sequence information on a gene of the atopic dermatitis-associated factor, and then performing manipulations for construction of an expression plasmid, transfection into a host, culture of a transformant, and collection of a protein from the culture. These manipulations can be performed according to a method known to a person skilled in the art, or a method described in literatures (Molecular Cloning, T. Maniatis et al., CSH Laboratory (1983), DNA Cloning, D M. Glover, IRL PRESS (1985)). Specifically, for example, a recombinant DNA (expression vector) capable of expressing a gene encoding the atopic dermatitis-associated factor in a desired host cell is made and then introduced into a host cell to transform it. The transformant is cultured, and the objective protein is collected from the resulting culture. The atopic dermatitis-associated factor can be also produced based on information of the amino acid sequence thereof by a general chemical synthesis method (peptide synthesis). Specifically, a solution phase synthesis method and a solid phase synthesis method described in “Basis and Experiment of Peptide Synthesis” (Nobuo Izumiya et al., Maruzen, Published in 1987) can be used.

The atopic dermatitis-associated factor utilized in the present invention includes not only a protein comprising the aforementioned amino acid sequence, but also homologues thereof. Examples of the homologue include proteins comprising the aforementioned amino acid sequence of the protein in which one or more amino acids are deleted, substituted or added, and having the biological function equivalent to the function of the protein, and/or having the immunological activity equivalent to the protein comprising the aforementioned amino acid sequence. Herein, “the immunological activity equivalent” includes a protein having the ability to bind specifically to an antibody directed to the atopic dermatitis-associated factor.

The “control” in the second step of the present assessing method means, for example,
(a) the case where a wild-type non-human animal which belongs to the same animal species as the present non-human animal used in the assessment method is subjected to the same steps as the first step and the second step, or
(b) the case where a control substance (positive control, negative control) in place of a test substance is used in the same steps as the first step and the second step.

In the case of the (a), when the atopic dermatitis treating effect or the atopic dermatitis development preventing effect in the present non-human animal is equivalent to or more than the atopic dermatitis treating effect or the atopic dermatitis development preventing effect of a test substance in the wild-type non-human animal, the test substance can be assessed to have an atopic dermatitis treating effect or an atopic dermatitis development preventing effect. When the atopic dermatitis treating effect or the atopic dermatitis development preventing effect in the present non-human animal is less than the atopic dermatitis treating effect or the atopic dermatitis development preventing effect of a test substance in the wild-type non-human animal, the test substance can be assessed not to have an atopic dermatitis treating effect or an atopic dermatitis development preventing effect due to the PHF3 protein.

In the case of the (b), examples of the control substance include a positive control and a negative control. The positive control refers to an arbitrary substance having an atopic dermatitis treating effect or an atopic dermatitis development preventing effect and, specific example thereof is corticosteroid or the like. Examples of the negative control include a solvent contained in a test substance, a test system solution which is a background, and the like.

In the case of the negative control as the control substance, when the atopic dermatitis treating effect or the atopic dermatitis development preventing effect of a test substance is more than the atopic dermatitis treating effect or the atopic dermatitis development preventing effect of the control substance, the test substance can be assessed to have an atopic dermatitis treating effect or an atopic dermatitis development preventing effect. On the other hand, when the atopic dermatitis treating effect or the atopic dermatitis development preventing effect of a test substance is equivalent to or less than the atopic dermatitis treating effect or the atopic dermatitis development preventing effect of the control substance, the test substance can be assessed not to have an atopic dermatitis treating effect or an atopic dermatitis development preventing effect.

In the case of the positive control as the control substance, the extent of the atopic dermatitis treating effect or the atopic dermatitis development preventing effect of a test substance can be assessed by comparing the atopic dermatitis treating effect or the atopic dermatitis development preventing effect of a test substance with the atopic dermatitis treating effect or the atopic dermatitis development preventing effect of the control substance.

Since substances (atopic dermatitis controlling substances) selected based on the atopic dermatitis treating effect or the atopic dermatitis development preventing effect of test substances determined by the present assessing method have an atopic dermatitis treating effect or an atopic dermatitis development preventing effect, they can be used as drugs or foods effective for treatment or prevention of a diseases with the development of atopic dermatitis or the like. Further, compounds derived from substances obtained by the present screening method can be also used similarly.

Substances obtained by the present screening method may form salts. Examples of the salt of the substance include salts with physiologically acceptable acids (e.g. inorganic acids, organic acids) or bases (e.g. alkali metal), and inter alia, physiologically acceptable acid addition salts are preferred. Examples of such salt include salts with inorganic acids (e.g. hydrochloric acid, phosphoric acid, hydrobromic acid, sulfuric acid), and salts with organic acids (e.g. acetic acid, formic acid, propionic acid, fumaric acid, maleic acid, succinic acid, tartaric acid, citric acid, malic acid, oxalic acid, benzoic acid, methanesulfonic acid, benzenesulfonic acid). Substances obtained by the present screening acid can be used orally as optionally sugar-coated tablets, capsules, elixirs, microcapsules or the like, or parenterally as the form of injectables such as sterile solutions or suspensions with water or other pharmaceutically acceptable liquid. These preparations can be prepared by mixing the substance with a physiologically acceptable carrier, flavor, excipient, vehicle, antiseptic, stabilizer, binder and the like in a unit dosage form required in generally recognized pharmaceutical practice. The amount of an active ingredient in these preparations is such an amount that a suitable volume in the indicated range is obtained. Examples of an additive which can be incorporated into a tablet, a capsule or the like include binders such as gelatin, corn starch, tragacanth, and gum arabic; excipients such as crystalline cellulose; swelling agents such as corn starch, gelatin, alginic acid and the like; lubricants such as magnesium stearate; sweeteners such as sugar, lactose and saccharine; flavors such as peppermint, Gaultheria adenothrix oil (akamono oil) and cherry, and the like. When the dosage unit form is a capsule, it may contains a liquid carrier such as a fat or oil in addition to the aforementioned type of materials. A sterile composition for injection can be formulated according to conventional pharmaceutical practice, for example by dissolving or suspending an active substance in a vehicle such as water for injection, and a naturally occurring vegetable oil such as a sesame oil, a palm oil or the like. As an aqueous liquid for injection, a physiological saline, an isotonic solution containing glucose or other auxiliary agent (e.g. D-sorbitol, D-mannitol etc.) is used, and may be used in combination with an alcohol (e.g. ethanol), a polyalcohol (e.g. propylene glycol, polyethylene glycol), a nonionic surfactant (e.g. Polysorbate 80 TM, HCO-50) or the like. As an oily liquid, for example, a sesame oil, a soybean oil or the like is used, and may be used in combination with benzyl benzoate, benzyl alcohol or the like which is a solubilizer. In addition, the aforementioned remedies or development prophylactics may contain, for example, a buffer (e.g. phosphate buffer, sodium acetate buffer), a soothing agent (e.g. benzalkonium chloride, procaine hydrochloride etc.), a stabilizer (e.g. human serum albumin, polyethylene glycol etc.), a preservative (e.g. benzyl alcohol, phenol etc.), an antioxidant and the like. The prepared pharmaceutical composition such as an injectable is usually filled into a suitable ample.

Since the thus obtained preparations are safe and low toxic, they can be administered to a human or a mammal (e.g. rat, rabbit, sheep, pig, cow, cat, dog, monkey etc.). A dose of the substance varies depending on a disease to be treated, a subject of administration, a route of administration and the like. For example, when the compound is orally administered, generally, the substance is administered in an amount of about 0.1 to 100 mg, preferably about 1 to 50 mg, more preferably about 1 to 20 mg per day to an adult (weight 60 kg). When parenterally administered, a dose per time of the substance varies depending on a subject of administration, a disease to be treated and the like. For example, when the substance is usually administered to an adult (weight 60 kg) in the form of an injectable, the substance is conveniently administered in an amount of about 0.01 to 30 mg, preferably about 0.1 to 20 mg, more preferably about 0.1 to 10 mg per day via intravenous injection. In the case of other non-human animals, an equivalent amount calculated on the basis of the aforementioned dose per 60 kg body weight can be administered.

The present invention further provides use of:

(1) the PHF3 protein or a partial region thereof;

(2.) a polynucleotide comprising a nucleotide sequence encoding the amino acid sequence of the protein or a partial region thereof described in (1), or a nucleotide sequence complementary to the nucleotide sequence; and

(3) a DNA encoding a RNA having a ribozyme activity which cleaves specifically a nucleotide sequence encoding the amino acid sequence of the protein or a partial region thereof described in (1); and

(4) a DNA encoding a RNA which suppresses the expression of the PHF3 gene on the basis of RNAi effect when expressed in a cell, for protein therapy or gene therapy of an allergy disease.

Herein, preferable examples of the PHF3 protein can include proteins which have an activity for ameliorating allergy disease symptoms and comprise any one of the following amino acid sequences:

<Amino Acid Sequence>

(a) the amino acid sequence of SEQ ID NO: 1 or 7;

(b) the amino acid sequence of SEQ ID NO: 1 or 7 in which one or more amino acids are deleted, added or substituted;

(c) an amino acid sequence with 75% or more sequence identity to the amino acid sequence of SEQ ID NO: 1 or 7;

(d) the amino acid sequence encoded by the nucleotide sequence of SEQ ID NO: 2 or 8;

(e) an amino acid sequence encoded by a DNA comprising a nucleotide sequence with 80% or more sequence identity to the nucleotide sequence of SEQ ID NO: 2 or 8; and

(f) an amino acid sequence encoded by a DNA which hybridizes with a DNA comprising a nucleotide sequence complementary to the nucleotide sequence of SEQ ID NO: 2 or 8 under stringent conditions, and the like.

As shown in Examples described hereinbelow, since the PHF3 gene is highly expressed in human leukocytes and the expression is reduced by stimulation with a mitogen, the activation state of a leukocyte and the PHF3 gene expression have a close relationship. Therefore, introduction of the protein, the polynucleotide or the like into a body results in a compensated expression amount of the PHF3 gene, so that improvement in the symptoms of an allergy disease is expected.

When the PHF3 protein or the like is used as a drug, a protein whose antigenecity to a human is the lowest among the PHF3 protein and the like and which has the amino acid sequence of the PHF3 protein or at least a part thereof is preferably used as an active ingredient of a pharmaceutical composition.

The PHF3 protein can be prepared as a natural protein from a naturally-occurring organism by manipulation such as extraction and purification, or can be prepared as a recombinant protein using a genetic engineering procedure. For example, a crude extract can be prepared from a human cell or tissue, and then purified using a variety of columns to prepare the purified protein. Herein, the cell is not particularly limited as long as it is producing the PHF3 protein and, for example, a leukocyte-derived cell or the like can be used.

Further, based on a nucleotide sequence of the PHF3 gene, an antisense drug can be developed. That is, a part of the PHF3 gene or a derivative thereof synthesized by a known method can used to regulate the expression of the PHF3 protein. An oligonucleotide comprising a sequence complementary to the PHF3 gene or a derivative thereof can be also used to regulate the expression of the PHF3 protein.

The present invention further provides use of:

(1) the PHF3 protein or a partial region thereof;

(2) a polynucleotide comprising a nucleotide sequence encoding the amino acid sequence of the protein or a partial region thereof described in (1), or a nucleotide sequence complementary to the nucleotide sequence; and

(3) a gene of the PHF3 protein, for diagnosing atopic dermatitis.

Herein, preferable examples of the PHF3 protein include proteins which have an activity for ameliorating allergy disease symptoms and comprise any one of the following amino acid sequences:

<Amino Acid Sequence>

(a) the amino acid sequence of SEQ ID NO: 1 or 7;

(b) the amino acid sequence of SEQ ID NO: 1 or 7 in which one or more amino acids are deleted, added or substituted;

(c) an amino acid sequence with 75% or more sequence identity to the amino acid sequence of SEQ ID NO: 1 or 7;

(d) the amino acid sequence encoded by the nucleotide sequence of SEQ ID NO: 2 or 8;

(e) an amino acid sequence encoded by a DNA comprising a nucleotide sequence with 80% or more sequence identity to the nucleotide sequence of SEQ ID NO: 2 or 8; and

(f) an amino acid sequence encoded by a DNA which hybridizes with a DNA comprising a nucleotide sequence complementary to the nucleotide sequence of SEQ ID NO: 2 or 8 under stringent conditions.

Herein, the gene of the PHF3 protein refer to those having a broad meaning, including in addition to a nucleotide sequence encoding the protein described above in (1), a transcription regulating region present upstream of the nucleotide sequence.

As described above, since the PHF3 gene is highly expressed in human leukocytes and the expression is reduced by stimulation with a mitogen, the activation state of a leukocyte and the PHF3 gene expression have a close relationship. Therefore, the expression amount of the PHF3 gene can be an index for diagnosis of atopic dermatitis. The aforementioned diagnosis of atopic dermatitis includes, for example, so-called genetic diagnosis (e.g. including mRNA level diagnosis, genome level diagnosis etc.) for detecting susceptibility, and provides use relating to technique including also a method of detecting a genetic mutation or gene polymorphism in the gene of the PHF3 protein or a transcription regulating region of the gene, as a means for diagnosing sensitivity of an allergy disease.

Specific examples of such method include a hybridization method using a probe of a DNA which has a nucleotide sequence of the gene of the PHF3 protein and is labeled with an enzyme such as horseradish peroxidase (HRP) or the like, a radioactive isotope element, a fluorescent substance, a chemiluminescent substance or the like, and a PCR method using primers which is designed based on a nucleotide sequence of the DNA.

Herein, the physiological activity relating to a transcription regulating region of the gene of the PHF3 protein may be assessed, for example, by a reporter gene assay method which comprises making a recombinant DNA molecule in which a suitable reporter gene is connected to the downstream of a DNA comprising the nucleotide sequence of the transcription regulating region, transforming a suitable host cell with the recombinant DNA molecule, and then detecting and quantitating a reporter gene product produced from the resulting transformant, a known gel shift assay method using a labeled DNA comprising the nucleotide sequence of the transcription regulating region, or the like. Such assay method can be also applied for screening of substances which inhibit or activate the expression of the PHF3 gene.

### EXAMPLES

The present invention will be explained below by Examples, but the present invention is not limited to them.

### Example 1

### Preparation of PHF3 Gene Knockout ES Cell

PHF3 gene knockout ES cells were prepared by a gene trap method (International Publication WO01/005987) using a trap vector pU-17.

It was confirmed that a disrupted or recombined gene is only the PHF3 gene, according to the following method.

In the ES cell used, it was confirmed by PCR analysis that only a copy of the vector was introduced, and it was also confirmed by Southern blotting analysis that the full length of the vector was introduced. Further, when a partial sequence of the trapped gene (a cDNA sequence upstream of an insertion position of the trap vector) was determined by a 5′ RACE method, it was consistent with the nucleotide sequences of the first exon and the second exon of the PHF3 gene (GenBank Acc. No. XM_{—}129836) (see FIG. 1). The mouse PHF3 gene is composed of 16 exons, and encodes a protein consisting of 2025 amino acids. A translation initiation codon of the gene is present in the second exon. In a cell in which the trap vector pU-17 is inserted immediately after the second exon, a protein in which the PHF3 protein is fused with βgeo immediately after the start of translation is translated, and the original PHF3 protein is not made. Then, the nucleotide sequence of the genome gene of the ES cell used was analyzed. As a result, it was confirmed that the trap vector pU-17 was inserted between the second exon and the third exon. Thereby, it was found that the ES cell was a cell deficient in the expression of the PHF3 gene due to alteration of the gene on a chromosome.

### Example 2

### Production of PHF3 Gene Knockout Mouse

A chimeric mouse comprising cells in which the PHF3 gene was knocked out was produced according to the following method. The ES cell prepared in Example 1 in which the PHF3 gene was disrupted or recombined and an ICR line 8-cell stage embryo were aggregated by an aggregation method (Nagy A., 1993, Oxford University Press, Oxford, pp 147-179). On the following day, the chimeric embryo which had been developed into a morula or a blastocyst was transplanted into the uterus of a pseudo-pregnancy-induced ICR line female mouse (pseudo-pregnancy induction day 3) (Shinichi Aizawa: Gene targeting: Preparation of Mutated Mouse using ES Cell, Yodosha Co., Ltd, 1995). After 17.5 days from transplantation, the female mouse was subjected to caesarotomy to take out a fetal, and thereby a chimeric mouse was obtained.

The chimeric mouse was reared by a foster parent for 4 weeks (Shinichi Aizawa: Gene targeting: Preparation of Mutated Mouse using ES Cell, Yodosha Co., Ltd, 1995). The chimeric mouse was weaned at 4 week, and mated with two ICR line female mice at 6 week to confirm germline transmission (GT). A tissue of a baby born between the chimeric mouse and ICR was taken. The genome DNA of the tissue was subjected to PCR analysis to confirm the presence or absence of the introduced gene, and subjected to Southern analysis to confirm whether the gene of the ES cell and the gene of the mouse are consistent or not. The chimeric mouse for which GT determination had been completed was mated naturally with two C57BL/6J female mice to produce F1 heterozygotes of the PHF3 gene knockout mouse.

Fertilized eggs obtained by in vitro fertilization between the produced F1 heterozygous male knockout mouse and a hyperovulation-treated C57BL/6J female mouse were transplanted into a pseudo-pregnancy-induced ICR line female mouse (recipient) to produce a large number of F2 heterozygous knockout mice. IVF was performed using the produced F2 heterozygous knockout mouse to produce fertilized eggs. The embryo was transplanted into a recipient to produce a F3 homozygous knockout mouse.

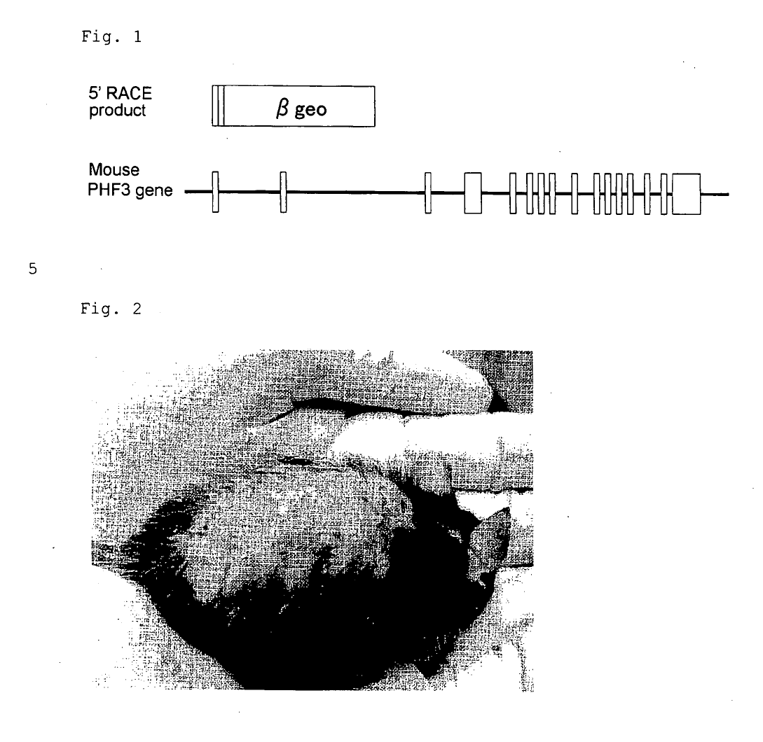

The appearance of the thus produced PHF3 gene homo-knockout mouse (33 week-old) was observed. An example is shown in FIG. 2. In the mouse, hair loss, formation of scabs over a wide range of the back part and itching behavior were observed, which means atopic dermatitis symptoms were recognized.

### Example 3

### Pathological Test of PHF3 Gene Homo-Knockout Mouse

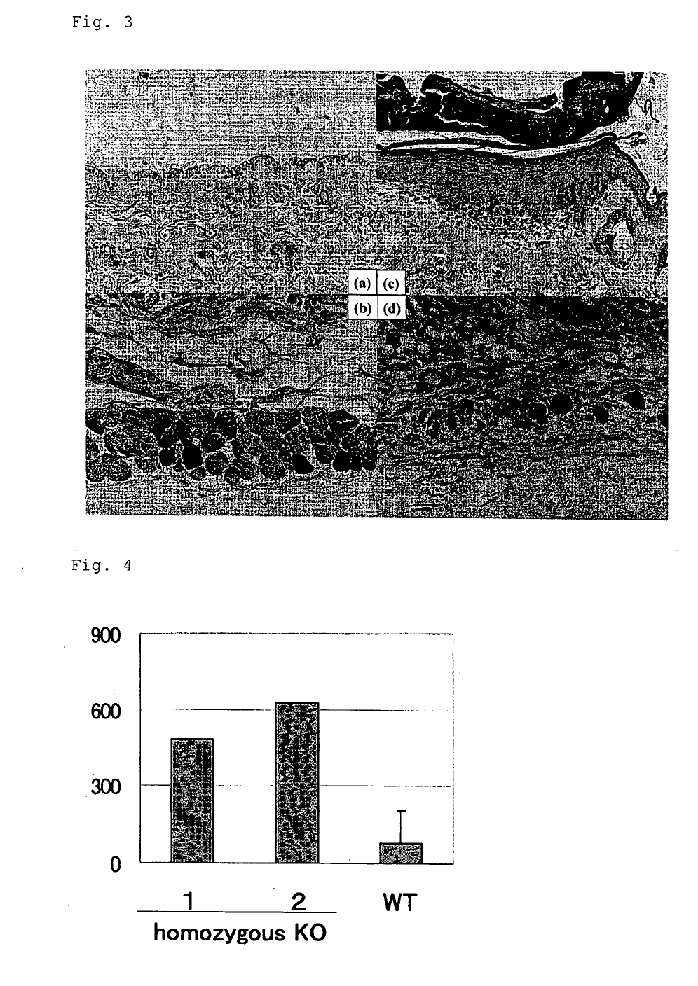

The atopic dermatitis symptoms found in the PHF3 gene homo-knockout mouse produced in Example 2 were histopathologically tested according to the following method. After the 8 week-old PHF3 gene homo-knockout mouse was led to death by exsanguination under ether anesthesia, a part of a skin tissue (abdominal) was taken from the mouse and fixed in a 10% neutral buffered formalin solution at room temperature for 1 week. The fixed skin tissue was alcohol-dehydrated, then embedded in paraffin, and thinly sliced at a thickness of 2 to 10 um using a microslicer. The resulting thin section was placed on a slide glass. The thin section was de-paraffined (7 min×3) with xylene, washed with 100% ethanol (30 sec×1, 2 min×2) and then distilled water, and then stained with hematoxylin/eosin (hereinafter, referred to as HE staining). The HE staining was performed by immersing the section in a hematoxylin solution (a solution of hematoxylin 2 g, aluminum potassium sulfate 75 g, sodium iodate 0.4 g, citric acid 1 g and chloral hydrate 50 g in 1 L of distilled water) for 10 minutes, washing the section with distilled water for 15 minutes, immersing the section in an eosin solution (a mixture of a 1% eosin aqueous solution 200 mL, a 1% phloxine B aqueous solution 5 mL and 80% ethanol 615 mL, which was adjusted to pH 5.5 with acetic acid) for 2 minutes, rinsing it with distilled water again for 15 second, and then dehydrating it sequentially with 80% ethanol (15 seconds), 90% ethanol (15 seconds), 100% ethanol (2 min×2) and xylene (5 min×3). Thereafter, the HE-stained skin tissue was subjected to a histopathological test using a light microscope. Results are shown in FIG. 3. In the PHF3 gene homo-knockout mouse, proliferation of prickle cells, formation of scabs, and cell infiltration into the skin dermis layer were observed and, additionally, cell infiltration into a subcutaneous tissue and atrophy of cutaneous muscle were observed. Thereby, it was confirmed that the PHF3 gene homo-knockout mouse exhibits atopic dermatitis symptoms.

### Example 4

### Measurement of Immunoglobulin E Level

The total immunoglobulin E (IgE) level in the serum of the PHF3 gene homo-knockout mouse was studied. About 40 μl of blood was taken from the tail vein of the mouse, and the serum was separated by a conventional method. The IgE level in the serum was measured with Lewis IgE-ELISA KIT (mouse) manufactured by Shibayagi Co., Ltd according to the method recommended by the kit. Results are shown in FIG. 4. The 43 week-old PHF3 gene homo-knockout mouse individual had a high level of total IgE in serum. To the contrary, the wild-type mouse had a low level of total IgE in serum. Therefore, it was found that the dermatitis caused by disruption or recombination of the PHF3 gene produces an increased level of IgE, which is a symptom peculiar to atopic dermatitis.

### Example 5

### Analysis of PHF3 Gene Expression in Leukocytes (In Vitro)

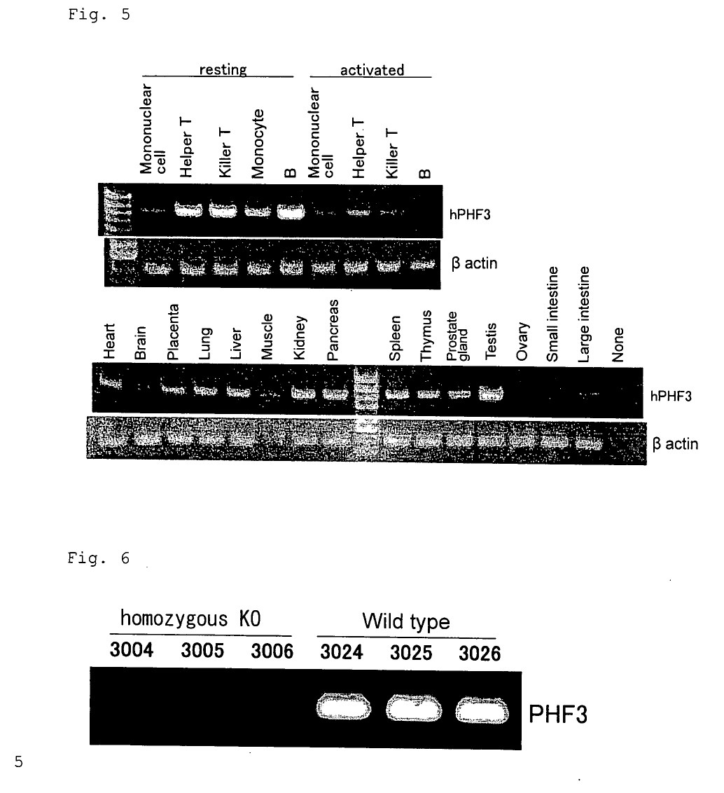

The presence or absence of PHF3 mRNA in an RNA derived from a human leukocyte specimen was studied by a reverse transcription-PCR method. PCR was performed for 32 cycles in a 25 μl reaction system using each 12.5 pmol of a primer set for specific amplification of the PHF3 protein, 1 μl of a human normal leukocyte (mononuclear cell, killer T cell, helper T cell, monocyte, B cell)-derived cDNA (manufactured by Clontech) as a template, and ExTaq DNA polymerase (manufactured by TAKARA SHUZO Co., Ltd.). Of the leukocyte samples, the samples marked with “resting” are cDNAs derived from hemocytes which were subjected to only a treatment of separation from blood, and the samples marked with “activated” are cDNAs derived from a mononuclear cell treated with concanavalin A (ConA) and pokeweed mitogen (PWM), a helper T cell treated with ConA, a killer T cell treated with phytohemagglutinin, or a B cell treated with PWM. In the PCR, hPHF3 F1 (SEQ ID NO: 3: gtggcaatga taccattgat ga) was used as a sense primer, and hPHF3 R2 (SEQ ID NO: 4: cacttcatct acacaactag gca) was used as an antisense primer. An amplified DNA from the PHF3 mRNA is a DNA having 821 base pairs. The amount of a β actin mRNA was also measured using the same template cDNA. PCR was performed for 25 cycles using β actin F1 (SEQ ID NO;5: accaactggg acgacatgga gaa) as a sense primer and β actin R1 (SEQ ID NO: 6: gtggtggtga agctgtagcc) as an antisense primer. An amplified DNA from a β actin mRNA is a DNA having 380 base pairs. Results are shown in FIG. 5.

The PHF3 mRNA was highly expressed in untreated (resting) leukocytes, while the expression amount was remarkably reduced in a helper T cell treated with concanavalin A, a killer T cell treated with phytohemagglutinin and B cell treated with pokeweed mitogen. The expression amount of β actin mRNA was approximately the same extent in each sample. From these results, it was found that the expression of PHF3 gene is suppressed in activated leukocyte cells.

### Example 6

### Analysis of Expression of PHF3 Gene in Knockout Mouse

It was studied by a PCR method that transcription of the PHF3 gene is suppressed in the PHF3 gene homo-knockout mouse produced by the present invention.

The submandibular lymph nodes of three PHF 3 gene homo-knockout mice (#3004-3006) and wild-type mice (#3024-3026) were removed. The total RNAs were prepared from the submandibular lymph nodes using RNeasy Mini kit (Quiagen). A cDNA was synthesized in a 30 μl reaction system from 0.6 μg of the total RNA using TaqMan Reverse Transcription Reagent (Applied Biosystems). PCR was performed in 25 μl of a reaction solution using a buffer attached to an enzyme. The reaction solution contained 1 μl of a template cDNA solution, 0.01 unit of KOD plus DNA polymerase (Toyobo Co., Ltd.), 0.5 μM of a sense primer mPHF3 F2 comprising a partial nucleotide sequence of exon 2 (SEQ ID NO: 9: cccaatttcc agatgccttg), and 0.5 μM of an antisense primer mPHF3 R2 comprising a nucleotide sequence complementary to exon 4 (SEQ ID NO: 10: gcatgcatgt ttgggatcaa). Performance of PCR comprised preincubation at 94° C. for 2 minutes, followed by 36 cycles of at 94° C. for 15 seconds, at 59° C. for 30 seconds and at 68° C. for 45 seconds. Results are shown in FIG. 6. In the reactions using the cDNAs from the wild-type mouse lymph nodes, a DNA with a length of 501 bp was amplified. On the other hand, in the cDNAs from the homo-knockout mice, a DNA was not amplified. It was confirmed that expression of the gene is suppressed due to insertion of a trap vector into the genome in the homo-knockout mice.

### INDUSTRIAL APPLICABILITY

According to the present invention can provide a knockout non-human animal whose genome comprises a disruption in at least one allele of the PHF3 gene, more specifically, a knockout non-human animal whose genome comprises a replacement of both or one allele of the diploid PHF3 gene with a function-deficient mutant gene or function-reduced mutant gene thereof, or such a disruption or recombination of both or one allele of the diploid PHF3 gene as to delete or reduce its function, or a progeny animal thereof or a part thereof; a totipotent cell such as an ES cell essential for production of the non-human animal or a part thereof; and a use thereof. The present invention is useful in the field of development of drugs or foods for a disease with the development of atopic dermatitis, and the like.

Free Text in Sequencing Listing

SEQ ID NO: 3
Designed oligonucleotide primer for PCR
SEQ ID NO: 4
Designed oligonucleotide primer for PCR
SEQ ID NO: 5
Designed oligonucleotide primer for PCR
SEQ ID NO: 6
Designed oligonucleotide primer for PCR
SEQ ID NO: 9
Designed oligonucleotide primer for PCR
SEQ ID NO: 10
Designed oligonucleotide primer for PCR

1. A knockout non-human animal whose genome comprises a disruption in at least one allele of the PHF3 gene.

2. The knockout non-human animal according to claim 1, wherein the disruption leads to an insufficient expression of the PHF3 gene.

3. The knockout non-human animal according to claim 1, wherein the disruption leads to the development of atopic dermatitis.

4. The knockout non-human animal according to claim 1, wherein the disruption prevents transcription of a full-length mRNA from the allele of the PHF3 gene.

5. The knockout non-human animal according to claim 1, wherein the disruption prevents transcription from exon 3 and the downstream region thereof in the allele of the PHF3 gene.

6. The knockout non-human animal according to claim 1, wherein the disruption comprises insertion of a gene cassette.

7. The knockout non-human animal according to claim 6, wherein the gene cassette comprises a nucleotide sequence encoding a selectable marker.

8. The knockout non-human animal according to claim 6, wherein the gene cassette comprises a nucleotide sequence encoding a selectable marker and a transcription termination signal.

9. The knockout non-human animal according to claim 1, wherein the disruption is a homozygous disruption.

10. The knockout non-human animal according to claim 1, wherein the non-human animal is a mammal.

11. The knockout non-human animal according to claim 1, wherein the non-human animal is a rodent.

12. The knockout non-human animal according to claim 1, wherein the non-human animal is a mouse.

13. The knockout non-human animal according to claim 12, wherein the PHF3 gene comprises a nucleotide sequence encoding the amino acid sequence of SEQ ID NO: 1.

14. The knockout non-human animal according to claim 1, wherein the disruption leads to representation of one or more properties selected from the group consisting of the following (a) to (d):
(a) high-frequency formation of scabs on the skin as compared with the wild-type non-human animal;
(b) high-frequency cell infiltration into the dermis layer of the skin as compared with the wild-type non-human animal;
(c) a high level of total immunoglobulin E in the blood as compared with the wild-type non-human animal; and
(d) a variation in the expression amount of an atopic dermatitis-associated factor as compared with the wild-type non-human animal.

15. A tissue or cell isolated from the knockout non-human animal according to claim 1.

16. The tissue or cell according to claim 15, which is a T cell, a B cell or a leukocyte-derived cell.

17. A cultured animal cell whose genome comprises a disruption in at least one allele of the PHF3 gene.

18. The cultured animal cell according to claim 17, wherein the disruption leads to an insufficient expression of the PHF3 gene.

19. The cultured animal cell according to claim 17, which is totipotent.

20. The cultured animal cell according to claim 17, wherein the PHF3 gene comprises a nucleotide sequence encoding the amino acid sequence of SEQ ID NO: 1.

21. The cultured animal cell according to claim 17, wherein the PHF3 gene comprises a nucleotide sequence encoding the amino acid sequence of SEQ ID NO: 7.

22. An animal model for a disease with the development of atopic dermatitis, comprising the non-human animal according to claim 1.

23. A method for analyzing a state of a disease associated with the PHF3 protein, which comprises performing on the non-human animal according to claim 1 during the period from the embryonic stage to death:
observation of growth and differentiation, development, and living behavior;
a histopathological test; or
a biochemical test.

24. The analyzing method according to claim 23, wherein the disease associated with the PHF3 protein is a disease with the development of atopic dermatitis.

25. A method for assessing an ability to control atopic dermatitis, which comprises:
(1) the first step of contacting the non-human animal according to claim 1 or a part thereof with a test substance,
(2) the second step of measuring an expression amount of an atopic dermatitis-associated factor in the non-human animal or a part thereof which has been contacted with the test substance in the first step, or an index value correlated with the expression amount, and then comparing the measured value with a control, and
(3) the third step of assessing an ability of the test substance to control atopic dermatitis on the basis of the comparison result of (2).

26. The assessing method according to claim 25, wherein the ability to control atopic dermatitis is an ability to control the expression of an atopic dermatitis-associated factor.

27. The assessing method according to claim 25, wherein the expression amount of an atopic dermatitis-associated factor is a RNA amount or a protein amount of an atopic dermatitis-associated factor.

28. The assessing method according to claim 25, wherein the atopic dermatitis associated factor is one or more factors selected from the group consisting of IFN-γ, IL-4, IL-5, IL-1 3, IL-18 and immunoglobulin E.

29. The assessing method according to claim 28, wherein the atopic dermatitis-associated factor is immunoglobulin E.

30. The assessing method according to claim 25, wherein the index value correlated with the expression amount of an atopic dermatitis-associated factor is an amount of formed scabs.

31. The assessing method according to claim 25, wherein the index value correlated with the expression amount of an atopic dermatitis-associated factor is a cell infiltration amount into the dermis layer of the skin.

32. The assessing method according to claim 25, wherein the index value correlated with the expression amount of an atopic dermatitis-associated factor is the total immunoglobulin E level in the blood.

33. A method for screening an atopic dermatitis controlling substance, which comprises selecting a test substance having an ability to control atopic dermatitis on the basis of an ability to control atopic dermatitis assessed by the assessing method according to claim 25.

34. A remedy or prophylactic for a disease with the development of atopic dermatitis, which comprises a substance having an ability to control atopic dermatitis obtained by the screening method according to claim 33 as an active ingredient.

35. A recombinant vector for producing the non-human animal according to claim 1, wherein the vector carries a gene cassette comprising a selectable marker gene from which a promoter region has been removed, and wherein the vector further comprises a nucleotide sequence homologous to a part of the nucleotide sequence of the PHF3 gene.

36. The recombinant vector according to claim 35, wherein the gene cassette is a gene cassette in which mouse EN2- derived SA (splicing acceptor), encephalomyocarditis Virus-derived IRES (internal ribosome entry site), βgeo in which β3-galactosidase and a neomycin resistant gene are fused, and SV40-derived pA (polyadenylation signal) are connected.

37. A method for protein therapy or gene therapy of an allergy disease, which comprises administering to a patient in need thereof a therapeutically effective amount of a substance selected from:
(1) the PHF3 protein or a partial region thereof;
(2) a polynucleotide comprising a nucleotide sequence encoding the amino acid sequence of the protein or a partial region thereof described in (1), or a nucleotide sequence complementary to the nucleotide sequence;
(3) a DNA encoding a RNA having a ribozyme activity which specifically cleaves a nucleotide sequence encoding the amino acid sequence of the protein or a partial region thereof described in (1); and
(4) a DNA encoding a RNA which suppresses the expression of the PHF3 gene on the basis of RNAi effect when expressed in a cell.

38. The use method according to claim 37, wherein the PHF3 protein has an activity for ameliorating allergy disease symptoms and comprises any one of the following amino acid sequences:
<Amino acid sequence>
(a) the amino acid sequence of SEQ ID NO: 1 or 7;
(b) the amino acid sequence of SEQ ID NO: 1 or 7 in which one or more amino acids are deleted, added or substituted;
(c) an amino acid sequence with 75% or more sequence identity to the amino acid sequence of SEQ ID NO: 1 or 7;
(d) the amino acid sequence encoded by the nucleotide sequence of SEQ ID NO: 2 or 8;
(e) an amino acid sequence encoded by a DNA comprising a nucleotide sequence with 80% or more sequence identity to the nucleotide sequence of SEQ ID NO: 2 or 8; and
(f) an amino acid sequence encoded by a DNA which hybridizes with a DNA comprising a nucleotide sequence complementary to the nucleotide sequence of SEQ ID NO: 2 or 8 under stringent conditions.

39. An antibody immunologically specific for a protein comprising the amino acid sequence of SEQ ID NO: 1 or 7 or a partial sequence thereof.

40. A method for diagnosing atopic dermatitis which comprises using a substance selected from:
(1) the PHF3 protein or a partial region thereof;
(2) a polynucleotide comprising a nucleotide sequence encoding the amino acid sequence of the protein or a partial region thereof described in (1), or a nucleotide sequence complementary to the nucleotide sequence; and
(3) a gene of the PHF3 protein.

41. The method according to claim 40, wherein the PHF3 protein has an activity for ameliorating allergy disease symptoms and comprises any one of the following amino acid sequences:
<Amino acid sequence>
(a) the amino acid sequence of SEQ ID NO: 1 or 7;
(b) the amino acid sequence of SEQ ID NO: 1 or 7 in which one or more amino acids are deleted, added or substituted;
(c) an amino acid sequence with 75% or more sequence identity to the amino acid sequence of SEQ ID NO: 1 or 7;
(d) the amino acid sequence encoded by the nucleotide sequence of SEQ ID NO: 2 or 8;
(e) an amino acid sequence encoded by a DNA comprising a nucleotide sequence with 80% or more sequence identity to the nucleotide sequence of SEQ ID NO: 2 or 8; and
(f) an amino acid sequence encoded by a DNA which hybridizes with a DNA comprising a nucleotide sequence complementary to the nucleotide sequence of SEQ ID NO: 2 or 8 under stringent conditions.
